(19) United States
(12) Patent Application Publication
(10) Pub. No.: US 2024/0225665 A1
(43) Pub. Date: Jul. 11, 2024

(54) SNAP-ON REAMER CONNECTOR

(71) Applicant: Zimmer, Inc., Warsaw, IN (US)

(72) Inventors: Joshua Clements, Roanoke, IN (US); Christopher A Gray, Warsaw, IN (US)

(21) Appl. No.: 18/405,566

(22) Filed: Jan. 5, 2024

Related U.S. Application Data
(60) Provisional application No. 63/437,864, filed on Jan. 9, 2023.

(51) Int. Cl. A61B 17/16; A61B 34/30

(52) U.S. Cl. CPC A61B 17/1613; A61B 34/30; A61B 2017/00477

(57) ABSTRACT

A reamer adapter may include a main body having a recess and a plurality of slots communicating with the recess. The reamer adapter may include at least a first spring coupled to the main body and positioned in one of the plurality of slots. The first spring can be configured to engage a cross-member of a reamer head and force the cross-member to engage with a part of the main body that forms the one of the plurality of slots.

### CLAIM OF PRIORITY

This application claims the benefit of U.S. Provisional Patent Application Ser. No. 63/437,864, filed on Jan. 9, 2023, the benefit of priority of which is claimed hereby, and which is incorporated by reference herein in its entirety.

### TECHNICAL FIELD

The present application pertains generally, but not by way of limitation, to orthopedic surgical reamers, and more specifically, to adapters for such reamers for coupling a head of the reamer with a shaft.

### BACKGROUND

It is often necessary to remove bone during orthopedic surgery to enable implantation of prosthesis to repair a joint of the human body. Reamers, for example, acetabular reamer cups and glenoid reamers are surgical tools which are used in surgery for the bone to prepare the joint to receive the prosthesis. Acetabular reamers have a head that is shaped as a cup. The head is used to cut hemispherical cavities in the pelvis bone for the implantation of an acetabular cup. Similarly, glenoid reamers are used to cut hemispherical cavities in the glenoid for the implantation of a prosthesis.

Adapters have been developed for coupling the head of the reamer with the shaft. These adapters are generally effective; however, they can be improved in one or more areas addressed by the adapters of the present application. For example, traditional adapters have multiple components including some that are expensive, some traditional adapters can only be operated in forward and reverse directions with difficulty, other traditional adapters are larger/bulky and/or are more difficult to clean than is desirable.

### Overview

The present inventors have recognized, among other things, adapters that address one or more of the problems discussed above. The present inventors have also recognized, among other things, that one of problems to be solved with traditional adapters can include reducing a profile of the head. The present inventors have recognized a low profile can be achieved with use of a single adapter component that is recessed within the reamer head itself via a basket type shape. The present inventors have recognized an adapter design that provides for positive locking force to keep the adapter and the head coupled together. The present inventors have also recognized, among other things, adapter designs that provide for equivalent forward and backward driving of the head (surgeon does not have to make modifications to the apparatuses or adjustment to technique), allows the head to be removable as desired (including in vivo) and allows for locking force/stiffness to be adjusted as desired. Traditional adapters typically do not provide for many of the above benefits/features recognized by the adapters of the present application.

The present inventors have also recognized, among other things, that the adapters disclosed herein can be used with robotic surgical systems including the ROSA® robot from Medtech, a Zimmer Biomet Holdings, Inc. company. This allows the benefits/features discussed above to be utilized with a robotic surgical system. Such surgical systems can allow for more precise control of reaming depth and location. In other words, the reamer center of rotation can be robotically controlled to ensure surgical accuracy.

### BRIEF DESCRIPTION OF THE DRAWINGS

FIG. 1 is a perspective view of a protype that is an assembly of an adapter coupled with a head of a reamer according to an example of the present application.

FIG. 2 provides a further prospective view of the assembly of FIG. 1 including an adapter coupled with a head of a reamer according to an example of the present application.

FIG. 2A is a side view of the assembly of FIG. 2.

FIG. 2B is a first cross-sectional view of the assembly of FIG. 2.

FIG. 2C is a second cross-sectional view of the assembly of FIG. 2.

FIG. 3A is a first side view of the adapter of FIG. 2.

FIG. 3B is a second side view of the adapter of FIG. 2.

FIG. 3C is a third side view of the adapter of FIG. 2.

FIG. 3D is a perspective view of the adapter of FIG. 2.

FIG. 3E is a plan view of a distal side of the adapter of FIG. 2.

FIG. 4 is a diagrammatic view of an operating room including a robot-assisted surgical system comprising a robotic arm, a computing system and a tracking system according to an example of the present application.

FIG. 5 is a schematic view of the robotic arm of FIG. 4 including an assembly of an adapter and a head of a reamer.

FIG. 6A is a first perspective view of the assembly of FIGS. 4 and 5.

FIG. 6B is a second perspective view of the assembly of FIGS. 4 and 5.

FIGS. 7A and 7B illustrate another adapter according to another example of the present application.

FIGS. 8A and 8B illustrate yet another adapter according to yet another example of the present application.

### DETAILED DESCRIPTION

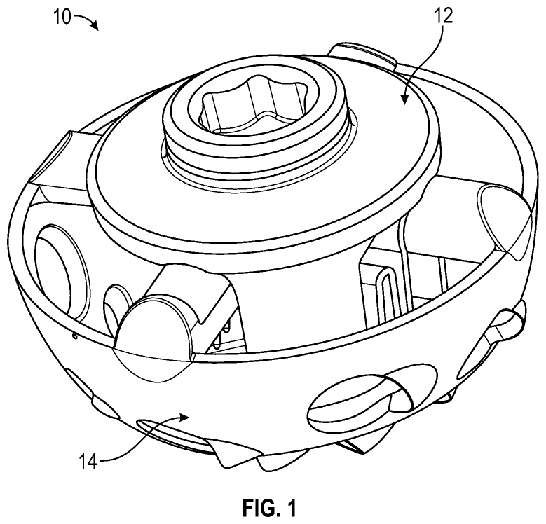

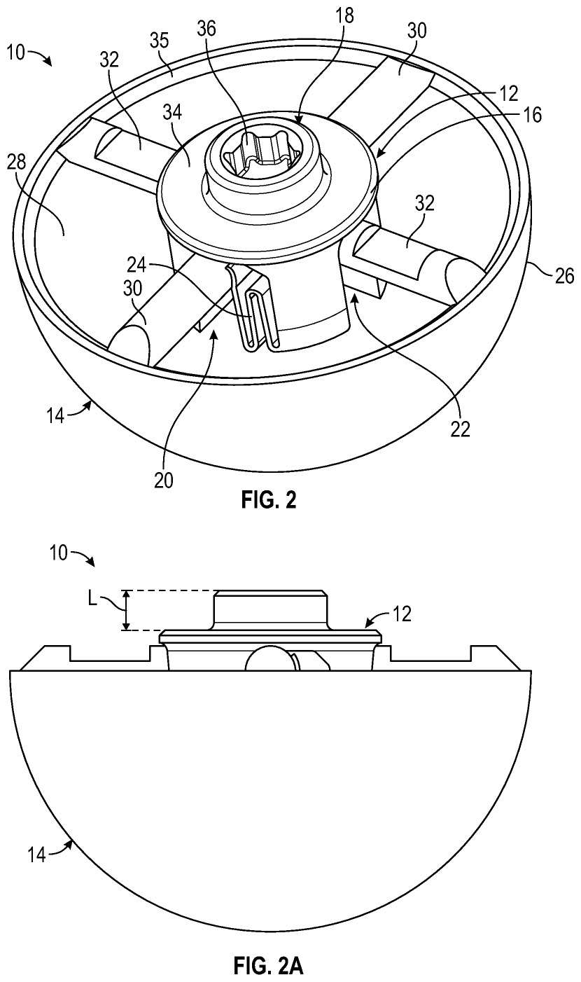

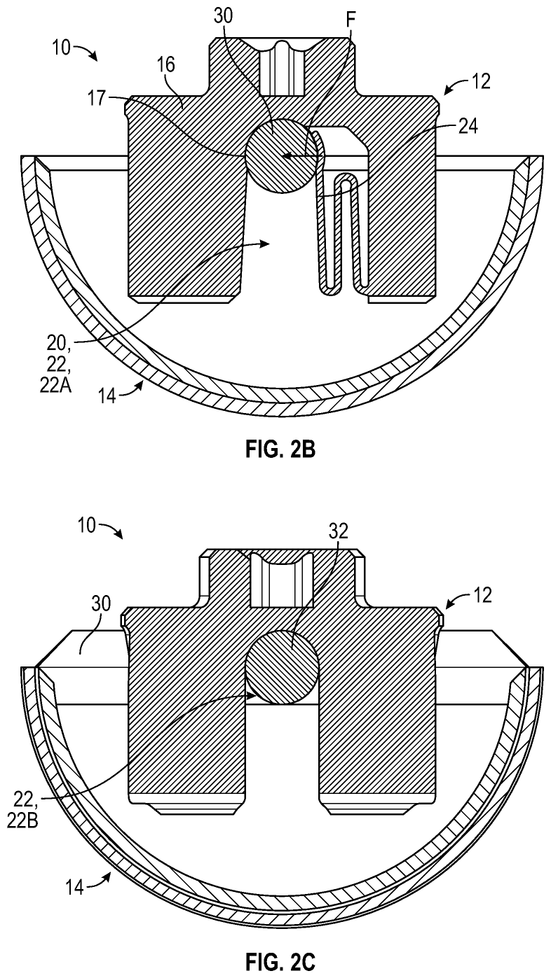

FIG. 1 illustrates an assembly 10 for a reamer. FIG. 2 additionally shows the assembly 10 for the reamer. The assembly 10 can include an adapter 12 and a reamer head 14 as shown in FIGS. 1-2C. Referring now to FIG. 2, the adapter 12 can include a main body 16, a first coupling feature 18, a recess 20, a plurality of slots 22 and a second coupling feature 24. The reamer head 14 can include an outer surface 26, an inner surface 28, a first cross-member 30 and a second cross-member 32.

The adapter 12 can be configured to seat down on and couple with the reamer head 14 as further described herein and illustrated. The adapter 12 can be configured to positively lock with the reamer head 14 by application of a continuous locking force via a force applied by the second coupling feature 24 as further discussed herein.

The main body 16 can be generally cylindrical in shape having a proximal surface 34. The main body 16 can be configured as a basket to receive parts of the reamer head 14 as further discussed herein. The main body 16 can extend distally from the proximal surface 34 and can be shaped to form the recess 20. The first coupling feature 18 can be connected to the main body 16 at the proximal surface 34. The first coupling feature 18 can extend proximally from the proximal surface 34 and/or can have portions extending distal of the proximal surface 34. The main body 16 can have an open frame design with an opening to the recess 20 on a distal side and the recess 20 centrally located distal of the proximal surface 34 and the first coupling feature 18. The plurality of slots 22 can be arranged around a circumference of the main body 16 and can provide openings to and can communicate with the recess 20. The second coupling feature 24 can be disposed at least partially within one or more of the plurality of slots 22 and/or the recess 20.

The reamer head 14 can have a hollow interior with a proximal opening capable of receiving distal portions of the main body 16. The outer surface 26 of the reamer head 14 can be generally hemispherical in shape and can include apertures and cutting features (see FIG. 1) for the removal of bone as known in the art. The inner surface 28 can be generally shaped to correspond with and be spaced by a wall from the outer surface 26. Thus, the inner surface 28 can be generally hemispherical in shape and can include the apertures (see FIG. 1) that pass through both the outer surface 26 and the inner surface 28. The first cross-member 30 and the second cross-member 32 can be located at or adjacent a proximal end 35 of the reamer head 14. The first cross-member 30 can be arranged transverse to the second cross-member 32 and can intersect with the second cross-member 32. This intersection can occur within the recess 20 of the adapter 12. In some examples, the first cross-member 30 and the second cross-member 32 can be an integral single piece component. However, other examples contemplate the first cross-member 30 and the second cross-member 32 can be separate components that are brazed, welded or otherwise joined. The first cross-member 30 can have a shape that differs from that of the second cross-member 32. The cylindrical shape of the first cross-member 30 can facilitate engagement by the second coupling feature 24 as shown in FIG. 2.

The main body 16 and the first coupling feature 18 can be formed of metal, metal alloy or another suitable material having sufficient strength and biocompatibility. The first coupling feature 18 can be a blind hex recess 36 or similar mating feature such as a thread, tab lock, stylet, etc. as known in the art. The blind hex recess 36 can be configured to mate with a corresponding male component of a shaft or another component, for example. The first coupling feature 18 can be configured to prevent pushing out or other relative movement of the reamer head 14 from shaft. The first coupling feature 18 can have a relatively small profile to accomplish coupling with the shaft or another adapter. This can be partially the result of the main body 16 extending into the reamer head 14. Thus, a proximal-distal length L (FIG. 2A) of the first coupling feature 18 can be between 2 mm and 6 mm from the proximal surface 34. The design of the adapter 12 can provide for a low profile reducing the amount of “nose” length needed for the connection between the reamer head 14 and the shaft.

FIG. 2B is a cross-section that illustrates the second coupling feature 24 engaging with the first cross-member 30 to form a positive lock. This positive lock is via force F due to the second coupling feature 24 being configured as a spring with a bias. The second coupling feature 24 forces the first cross-member 30 into engagement with a wall part 17 of the main body 16 that forms an edge of one of the plurality of slots 22. Engagement of the second coupling feature 24 with at least the first cross-member 30 can be facilitated by inserting the adapter 12 down on to the first cross-member 30 and the second cross-member (not shown) such that the plurality of slots 22 receive the first cross-member 30 and the second cross-member and the second coupling feature 24 is elastically deformed (flexed) by the first cross-member 30. Portions of the first cross-member 30 and the second cross-member can be received within the recess 20. The second coupling feature 24 with the force F can engage the first cross-member 30 and can force the first cross-member 30 into an engagement with the wall part 17 of the main body 16 that forms the edge of a first slot 22A of the plurality of slots 22.

As shown in the cross-section of FIG. 2C, the second cross-member 32 can be received in a second slot 22B of the plurality of slots 22. The second slot 22B can be arranged generally orthogonal to the first slot 22A. Although not shown in FIG. 2C, the present application anticipates further of the plurality of slots 22 can be shaped in a similar manner to the first slot 22A and the second slot 22B so as to receive other portions of the first cross-member 30 and the second cross-member 32, respectively. The configuration of the adapter 12 and the reamer head 14 particularly with the plurality of slots 22, the second coupling feature 24, the first cross-member 30 and the second cross-member 32 can allow for connection and disconnection of the adapter 12 from the reamer head 14 in relatively simple manner such as by snapping the second coupling feature 24 into and out of engagement with the first-cross member 30 as desired. Thus, the reamer head 14 can be removable from the adapter 12 in vivo, if desired by simply disengaging the second coupling feature 24 from the first cross-member 30.

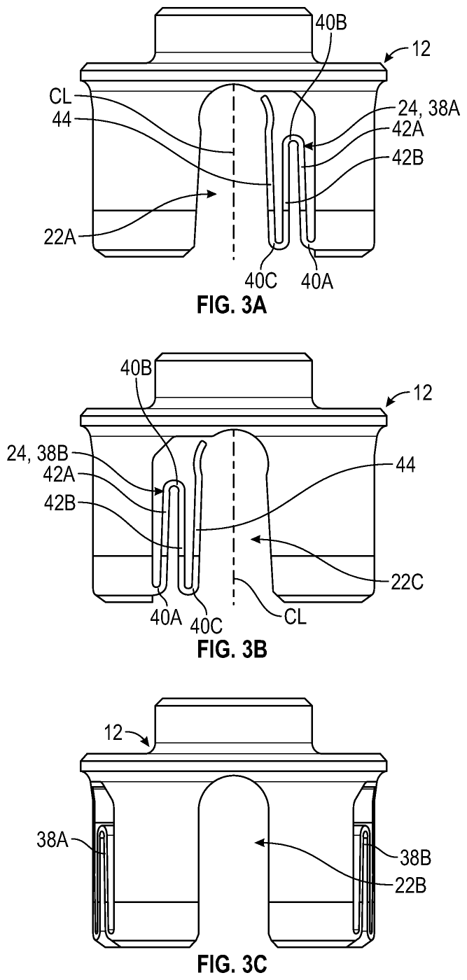

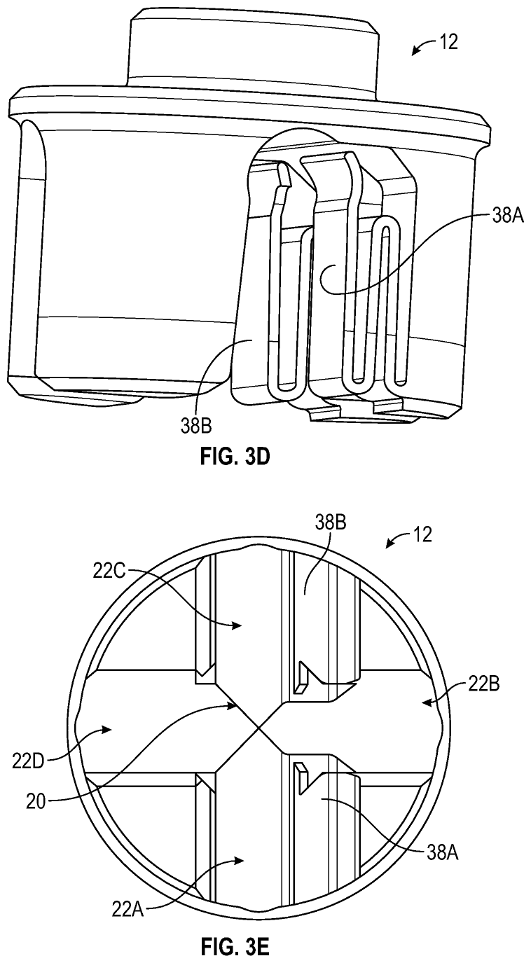

FIGS. 3A-3E show different views of the adapter 12. FIG. 3B shows an opposing side of the adapter 12 from FIG. 3A. According to the example of FIGS. 3A and 3B, the second coupling feature 24 can include a first spring 38A and a second spring 38B. However, other embodiments contemplate the second coupling feature 24 is a single spring or three or more springs, for example. FIG. 3A shows a first side with the first slot 22A and the first spring 38A positioned within the first slot 22A. FIG. 3C shows a second side with the second slot 22B as previously discussed in reference to FIG. 2C and shows both the first spring 38A and the second spring 38B. FIG. 3B shows a third side of the adapter 12 with a third slot 22C and the second spring 38B. The second spring 38B can be positioned within the third slot 22C. The third slot 22C and the second spring 38B can have substantially a mirror symmetry or similar shape to the first slot 22A and the first spring 38A, respectively. The second slot 22B and a fourth slot 22D (FIG. 3E) can be positioned generally orthogonal to the first slot 22A and the third slot 22C.

The first spring 38A and the second spring 38B can be formed of a same material as the main body 16 (e.g., stainless steel, titanium alloy, etc.). Thus, the main body 16, the first spring 38A and the second spring 38B can be a single piece integral component. The first spring 38A and the second spring 38B can be formed from the main body 16 such as by wire electrical discharge machining (EDM) or other machining or forming technique. According to some examples the first spring 38A and the second spring 38B can be 3D printed with the main body 16. The use of the first spring 38A and the second spring 38B in combination with the second slot 22B (and the fourth slot 22D shown in FIG. 3E) allow for both forward and back driving of the reamer head with a same stiffness in either direction. This same stiffness is provided by the identical shape of the second slot 22B and the fourth slot 22D. The second slot 22B and the fourth slot 22D are also symmetrically shaped to receive the second cross-member 32 (see FIG. 2C) and be engaged in either forward or back driving of the shaft.

The first spring 38A and the second spring 38B can include a first bend 40A, a second bend 40B, a third bend 40C, a first section 42A, a second section 42B and an engagement arm 44.

The first spring 38A and the second spring 38B can be connected to the main body 16. According to some examples, the first spring 38A and the second spring 38B can be machined or otherwise formed from the main body 16 so as to integral therewith. The first bend 40A can be positioned immediately adjacent or at the connection between the first spring 38A and the second spring 38B and the main body 16. The first bend 40A can comprise a turn of the first spring 38A and the second spring 38B from a first direction to a second direction. The first section 42A connects with the first bend 40A. The first section 42A extends proximally and radially inward toward center line CL of the main body 16 from the first bend 40A. The first section 42A can connect with the second bend 40B at a proximal end. The second bend 40B can provide for a change of direction between segments of the first spring 38A and the second spring 38B. Thus, the second bend 40B can be connected with both the first section 42A and the second section 42B. The second section 42B can extend distally from the second spring 38B to the third bend 40C. The engagement arm 44 can be connected to the third bend 40C and can extend proximally and radially inward toward center line CL of the main body 16 from the third bend 40A. A proximal end portion of the engagement arm 44 can be shaped (e.g., having a cylindrical indent, recess, bend or curvature) to engage with the first cross-member 30 as previously illustrated in FIG. 2B.

The number of bends and number of sections utilized for the first spring 38A and the second spring 38B is purely exemplary and illustrates only one possible configuration for the first spring 38A and the second spring 38B. The orientation of the first bend 40A, the second bend 40B, the third bend 40C, the first section 42A, the second section 42B and/or the engagement arm 44 can be adjustable via bending of the first spring 38A and the second spring 38B in some cases such as intraoperatively. This can be done via screw driver or another tool. This adjustment of orientation can allow for stiffness adjustment as the amount of interference of the first spring 38A and the second spring 38B with the first-cross member 30 can be adjusted. The configuration of the first spring 38A and the second spring 38B can also be modified during manufacture to achieve a locking force as desired by modifying the number of bends, the orientation of one or more the first bend 40A, the second bend 40B, the third bend 40C, the first section 42A, the second section 42B and/or the engagement arm 44, or other geometry.

For coupling of the first spring 38A and the second spring 38B with the first cross-member 30, the adapter 12 can be aligned such that the first cross-member 30 is generally aligned with the first and third slots 22A and 22C. Such alignment can align the second cross-member 32 with the second slot 22B (FIG. 3C) and the fourth slot 22D (FIG. 3E). The reamer head and/or adapter 12 can then be moved relative to one another such that the first cross-member 30 and the second cross-member 32 are received by the plurality of slots 22 in the manner previously illustrated in FIGS. 2-2C.

The first cross-member 30 can be engaged by first spring 38A and forced into engagement with part of the main body that forms part of the first slot 22A. A similar arrangement is accomplished by the second spring 38B. The second spring 38B can engage with another part of the first cross-member 30 and force the first cross-member 30 into engagement with part of the main body that forms part of the third slot 22C. The second cross-member 32 can be received by the second slot 22B and the fourth slot 22D (FIG. 3E) with relatively little play.

FIG. 3D shows a perspective view of the adapter 12 showing both the first spring 38A and the second spring 38B. FIG. 3E is a plan view of a distal side of the adapter 12 showing the recess 20, the first slot 22A, second slot 22B, the third slot 22C, the fourth slot 22D, the first spring 38A and the second spring 38B. FIG. 3E illustrates the recess 20, the first slot 22A, second slot 22B, the third slot 22C and the fourth slot 22D having openings to the distal side to allow for entry of the cross-members as previously illustrated and discussed.

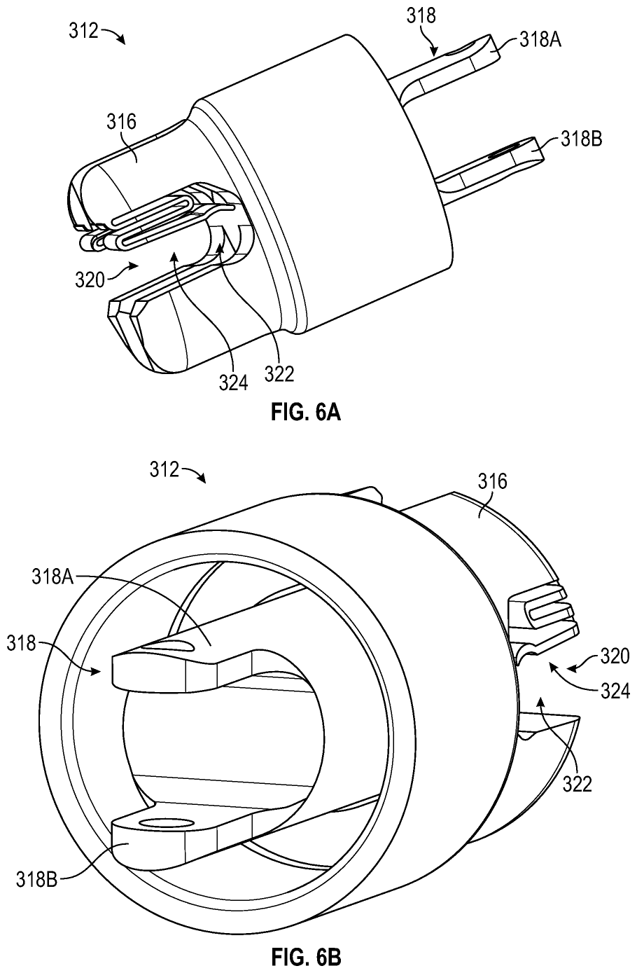

The adapter 12 can be modified for use with a robotic surgical system as further described herein. An exemplary version of the adapter that can be used with the robotic surgical systems discussed herein is illustrated in FIGS. 6A and 6B.

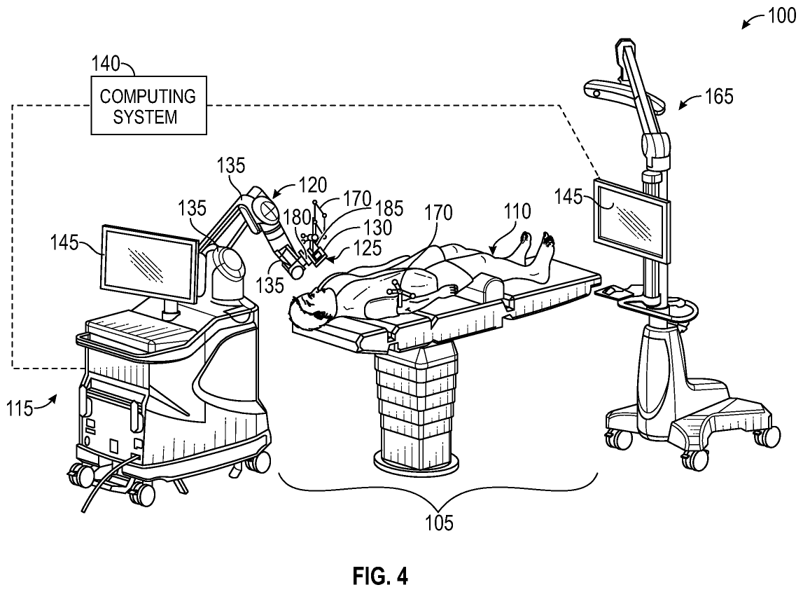

FIG. 4 illustrates surgical system 100 for operation on surgical area 105 of patient 110 in accordance with at least one example of the present disclosure. Surgical area 105 in one example can include a joint and, in another example, can be a bone. Surgical area 105 can include any surgical area of patient 110, including but not limited to the shoulder, head, elbow, thumb, spine, and the like. Surgical system 100 can also include robotic system 115 with one or more robotic arms, such as robotic arm 120. As illustrated, robotic system 115 can utilize only a single robotic arm. Robotic arm 120 can be a 6 degree-of-freedom (DOF) robot arm, such as the ROSA® robot from Medtech, a Zimmer Biomet Holdings, Inc. company. In some examples, robotic arm 120 is cooperatively controlled with surgeon input on the end effector or surgical instrument, such as surgical instrument 125. In other examples, robotic arm 120 can operate autonomously. While not illustrated in FIG. 4, one or more positionable surgical support arms can be incorporated into surgical system 100 to assist in positioning and stabilizing instruments or anatomy during various procedures.

Each robotic arm 120 can rotate axially and radially and can receive a surgical instrument, or end effector, 125 at distal end 130. Surgical instrument 125 can be any surgical instrument adapted for use by the robotic system 115, including, for example, a guide tube, a holder device, a gripping device such as a pincer grip, a burring device, a reaming device, an impactor device such as a humeral head impactor, a pointer, a probe, a collaborative guide or holder device as described herein or the like. Surgical instrument 125 can be positionable by robotic arm 120, which can include multiple robotic joints, such as joints 135, that allow surgical instrument 125 to be positioned at any desired location adjacent or within a given surgical area 105.

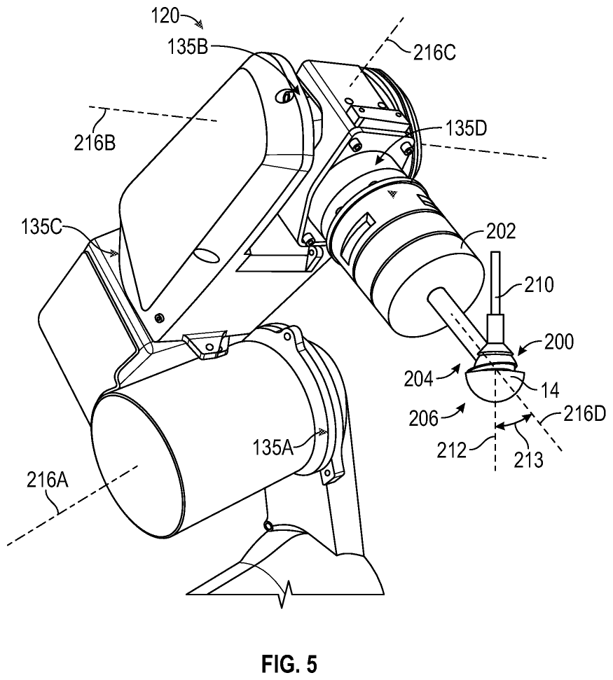

As discussed below, robotic arm 120 can be used with a reaming device, e.g., reaming system 200 (FIG. 5) that utilizes an adapter 312 (FIGS. 6A and 6B) and reamer head 14 as discussed herein, to direct the reamer head 14 along a controlled trajectory relative to surgical area 105 based on a virtual coordinate system determined by computing system 140, while still permitting a surgeon to manipulate the reamer head 14 within the parameters controlled by robotic arm 120.

Robotic system 115 can also include computing system 140 that can operate robotic arm 120 and surgical instrument 125. Computing system 140 can include at least memory, a processing unit, and user input devices, as will be described herein. Computing system 140 and tracking system 165 can also include human interface devices 145 for providing images for a surgeon to be used during surgery. Computing system 140 is illustrated as a separate standalone system, but in some examples computing system 140 can be integrated into robotic system 115. Human interface devices 145 can provide images, including but not limited to three-dimensional images of bones, glenoid, joints, and the like. Human interface devices 145 can include associated input mechanisms, such as a touch screen, foot pedals, or other input devices compatible with a surgical environment.

Computing system 140 can receive pre-operative, intra-operative and post-operative medical images. These images can be received in any manner and the images can include, but are not limited to, computed tomography (CT) scans, magnetic resonance imaging (MRI), two-dimensional x-rays, three-dimensional x-rays, ultrasound, and the like. These images in one example can be sent via a server as files attached to an email. In another example the images can be stored on an external memory device such as a memory stick and coupled to a USB port of the robotic system to be uploaded into the processing unit. In yet other examples, the images can be accessed over a network by computing system 140 from a remote storage device or service.

After receiving one or more images, computing system 140 can generate one or more virtual models related to surgical area 105. Alternatively, computer system 140 can receive virtual models of the anatomy of the patient prepared remotely. Specifically, a virtual model of the anatomy of patient 110 can be created by defining anatomical points within the image(s) and/or by fitting a statistical anatomical model to the image data. The virtual model, along with virtual representations of implants, can be used for calculations related to the desired location, height, depth, inclination angle, or version angle of an implant, stem, acetabular cup, glenoid cup, surgical instrument, or the like to be utilized in surgical area 105. In another procedure type, the virtual model can be utilized to determine insertion location, trajectory and depth for inserting an instrument. In a specific example, the virtual model can be used to determine a reaming angle relative to an acetabulum of a pelvis and a depth for reaming into the pelvis to place an acetabular implant. The virtual model can also be used to determine bone dimensions, implant dimensions, bone fragment dimensions, bone fragment arrangements, and the like. Any model generated, including three-dimensional models, can be displayed on human interface devices 145 for reference during a surgery or used by robotic system 115 to determine motions, actions, and operations of robotic arm 120 or surgical instrument 125. Known techniques for creating virtual bone models can be utilized, such as those discussed in U.S. Pat. No. 9,675,461, titled “Deformable articulating templates” or U.S. Pat. No. 8,884,618, titled “Method of generating a patient-specific bone shell” both by Mohamed Rashwan Mahfouz, as well as other techniques known in the art.

Computing system 140 can also communicate with tracking system 165 that can be operated by computing system 140 as a stand-alone unit. Surgical system 100 can utilize the Polaris optical tracking system from Northern Digital, Inc. of Waterloo, Ontario, Canada. Additionally, tracking system 165 can comprise the tracking system shown and described in Pub. No. US 2017/0312035, titled “Surgical System Having Assisted Navigation” to Brian M. May, which is hereby incorporated by this reference in its entirety. Tracking system 165 can monitor a plurality of tracking elements, such as tracking elements 170, affixed to objects of interest to track locations of multiple objects within the surgical field. Tracking system 165 can function to create a virtual three-dimensional coordinate system within the surgical field for tracking patient anatomy, surgical instruments, or portions of robotic system 115. Tracking elements 170 can be tracking frames including multiple IR reflective tracking spheres, or similar optically tracked marker devices. In one example, tracking elements 170 can be placed on or adjacent one or more bones of patient 110. In other examples, tracking elements 170 can be placed on robot robotic arm 120, surgical instrument 125, and/or an implant to accurately track positions within the virtual coordinate system associated with surgical system 100. In each instance tracking elements 170 can provide position data, such as patient position, bone position, joint position, robotic arm position, implant position, or the like.

Robotic system 115 can include various additional sensors and guide devices. For example, robotic system 115 can include one or more force sensors, such as force sensor 180. Force sensor 180 can provide additional force data or information to computing system 140 of robotic system 115. Force sensor 180 can be used by a surgeon to cooperatively move robotic arm 120. For example, force sensor 180 can be used to monitor impact or implantation forces during certain operations, such as insertion of an implant stem into a humeral canal. Monitoring forces can assist in preventing negative outcomes through force fitting components. In other examples, force sensor 180 can provide information on soft-tissue tension in the tissues surrounding a target joint. In certain examples, robotic system 115 can also include laser pointer 185 that can generate a laser beam or array that is used for alignment of implants during surgical procedures.

In order to ensure that computing system 140 is moving robotic arm 120 in a known and fixed relationship to surgical area 105 and patient 110, the space of surgical area 105 and patient 110 can be registered to computing system 140 via a registration process involving registering fiducial markers attached to patient 110 with corresponding images of the markers in patient 110 recorded preoperatively or just prior to a surgical procedure. For example, a plurality of fiducial markers can be attached to patient 110, images of patient 110 with the fiducial markers can be taken or obtained and stored within a memory device of computing system 140. Subsequently, patient 110 with the fiducial markers can be moved into, if not already there because of the imaging, surgical area 105 and robotic arm 120 can touch each of the fiducial markers. Engagement of each of the fiducial markers can be cross-referenced with, or registered to, the location of the same fiducial marker in the images. In additional examples, patient 110 and medical images of the patient can be registered in real space using contactless methods, such as by using a laser rangefinder held by robotic arm 120 and a surface matching algorithm that can match the surface of the patient from scanning of the laser rangefinder and the surface of the patient in the medical images. As such, the real-world, three-dimensional geometry of the anatomy attached to the fiducial markers can be correlated to the anatomy in the images and movements of instruments 125 attached to robotic arm 120 based on the images will correspondingly occur in surgical area 105.

Subsequently, other instruments and devices attached to surgical system 100 can be positioned by robotic arm 120 into a known and desired orientation relative to the anatomy. For example, robotic arm 120 can be coupled to a reaming system, such as reaming system 200 of FIG. 5, including a cooperatively positionable reamer of the present disclosure. Robotic arm 120 can move the reamer into a fixed position relative to anatomy of the patient such that an axis of the reamer extends along a desired orientation relative to the anatomy. With robotic arm 120 locked into place, the reaming systems of the present application can limit axial movement of the reamer to control reaming depth. However, the reaming system can also permit the surgeon to engage the reamer in multiple positions and to manipulate the reamer about a reamer pattern without moving off-axis or beyond the desired ream depth.

FIG. 5 is a schematic view of robotic arm 120 of FIG. 4 including reaming system 200, which can be positioned by robotic arm 120 relative to surgical area 105 (FIG. 4) in a desired orientation according to a surgical plan, such as a plan based on preoperative imaging. Reaming system 200 can comprise tool base 202, reaming guide 204 and reamer assembly 206. Reamer assembly 206 can comprise the reamer head 14 previously discussed, the adapter (discussed in reference to FIGS. 6A and 6B) and a reamer shaft 210, which can extend along axis 212.

Examples of reaming systems 200 that can be utilized with the adapters and reamer head 14 of the present application include U.S. application Ser. No. 17/185,529 published as 2021/0267609A1, entitled “SYSTEMS AND METHODS FOR CO-OPERATIVE CONTROL OF ROBOTICALLY-POSITIONED SURGICAL INSTRUMENTS” and U.S. application Ser. No. 17/747,635 published as 2022/0395343A1, entitled “QUICK CONNECT FOR ROBOTIC SURGICAL INSTRUMENTS”, each of which is hereby incorporated by reference in its entirety.

Robotic arm 120 can include joint 135A that permits rotation about axis 216A, joint 135B that can permit rotation about axis 216B, joint 135C that can permit rotation about axis 216C and joint 135D that can permit rotation about axis 216D. Reaming guide 204 can extend along a guide axis that can be coincident with axis 216D for joint 135D. Reamer axis 212 can be positioned at angle 213 relative to axis 216D via reaming guide 204. In examples, angle 213 can be in a range of approximately five degrees to approximately twenty-five degrees.

In order to position reaming system 200 relative to anatomy of patient 110 (FIG. 4), surgical system 100 (FIG. 4) can manipulate robotic arm 120 automatically by computing system 140 or a surgeon manually operating computing system 140 to move reaming system 200 to the desired location, e.g., a location called for by a surgical plan to align an instrument relative to the anatomy. For example, robotic arm 120 can be manipulated along axes 216A—216D to position reaming guide 204 such that reamer head 14 is located in a reaming location. With robotic arm 120 being immobilized (e.g., not moving) or locked in place, reaming guide 204 can position the center of reaming head 208 along a planned surgical trajectory and limit axial movement of reaming head beyond a planned surgical depth. However, reaming guide 204 can allow for tilting of reamer shaft 210 such that reamer head 14 can pivot about a center point to enable a surgeon to operate reamer 206 in an ergonomic manner and to move reamer head 14 in multiple, different passes across a bone surface to, for example, allow the surgeon to eliminate cutting tooth tracks in the bone surface. Thus, reamer shaft 210 can be moved to vary angle 213 relative to axis 216D.

Robotic arm 120 can be separately registered to the coordinate system of surgical system 100, such via use of a tracking element 170 (FIG. 4). Fiducial markers can additionally be separately registered to the coordinate system of surgical system 100 via engagement with a probe having a tracking element 170 attached thereto. Reaming guide 204 and reamer 206 can be registered to the coordinate system using trackers. As such, some or all of the components of surgical system 100 can be individually registered to the coordinate system and, if desired, movement of such components can be continuously or intermittently tracked with a tracking element 170.

It can be a difficult task to ensure instruments attached to robotic arm 120 are accurately aligned with and positioned relative to patient 110, particularly if instruments come in different sizes or the instrument needs to be individually manipulated during the procedure, such as by intervention of personnel including a surgeon. For example, sometimes robotic arm 120 is positioned to provide the proper alignment of an instrument, e.g., a reamer shaft or guide pin, that needs to be inserted into the patient. Thus, robotic arm 120 can automatically provide a trajectory for an instrument, while the surgeon manually provides the motive force for the instrument, e.g., rotation for a reamer shaft and insertion force for a guide pin. However, once the surgeon moves the instrument relative to robotic arm 120, the precise location of the instrument, e.g., the location of the tip of the instrument in the coordinate system, can become lost or obfuscated, and surgical system 100 will not be able to reproduce the location of said tip in imaging of the patient.

In some robotic procedures instruments can be separately tracked using an optical navigation system that, under ideal conditions, alleviate the need for precisely maintaining axis 212 and the location of an instrument along axis 212 through a surgical procedure or surgical task, as the optical navigation system can provide the surgical computer system information to compensate for any changes. However, as optical navigation systems require line-of-sight with the instruments to be maintained, there is a significant advantage in not requiring instruments to be navigated (or at least not constantly navigated). Accordingly, the ability to precisely maintain orientation of axis 212 and position along axis 212 provides the additional advantage of at least reducing, and possibly eliminating, the need to navigate instruments during a robotic procedure.

In order to improve the ability to determine the location of instruments within the coordinate system a registration device can be utilized. Thus, a trajectory or orientation of a reaming axis can be determined, as well as a reaming point along the reaming axis to ensure that a specific reamer shaft does not move beyond a planned surgical depth. After the registration process, the reaming axis orientation and reaming point can be continuously monitored via coupling to robotic arm 120 without requiring line-of-sight or specialty instruments, such that the position of the instrument relative to robotic arm 120 and the coordinate system can be determined.

The systems, devices and methods discussed in the present application can be useful in performing robotic-assisted surgical procedures that utilize robotic surgical arms that can be coupled to instrument holders used to precisely align trajectories of instruments relative to anatomy of a patient registered to the space of an operating room. The present disclosure describes adjustable instrument holders that can remain mounted to a robotic surgical arm throughout a surgical procedure and that can couple to a reamer adapter (e.g., the adapter 312 or the adapter 12) as disclosed and illustrated (e.g., in FIGS. 1-3B, 6A and 6B) herein.

FIGS. 6A and 6B show the adapter 312 that can be used with the reaming system 200 including the reamer head 14 (FIGS. 1-5). The adapter 312 can include a main body 316, a first coupling feature 318, a recess 320, a plurality of slots 322 and a second coupling feature 324.

The main body 316 can have a cylindrical basket shape as previously described in reference to FIG. 2 and can be modified slightly from the construct previously shown. The first coupling feature 318 can be modified to comprise flanges 318A and 318B for connection with feature(s) of the reamer shaft 210 (FIG. 5) or other components such as the instrument holder(s) coupled to the robotic surgical arm discussed previously. Modification to the first coupling feature 318 can be the primary difference between the adapter 312 and the adapter 12 discussed previously. The first coupling feature 318 (the flanges 318A and 318B) can extend proximally from a proximal backside of the main body 316. The recess 320 can be centrally located on a distal side of the main body 316 and can have openings including those formed by the plurality of slots 322. The plurality of slots 322 can communicate with the recess 320 in the manner previously described in FIGS. 2-3C. According to some examples, some of the plurality of slots 322 can be arranged orthogonal to one another and can be arranged and sized to receive the cross-members of the reamer head 14, which can be oriented substantially transverse to one another. The second connection member 324 (e.g., two springs) can be configured in the manner previously described in FIGS. 2-3C with the two springs arranged in two of the plurality of slots 322. The two springs are configured to engage with at least one of the cross-members of the reamer head 14 in the manner previously discussed to form a positive lock the engages the adapter 312 with the reamer head 14.

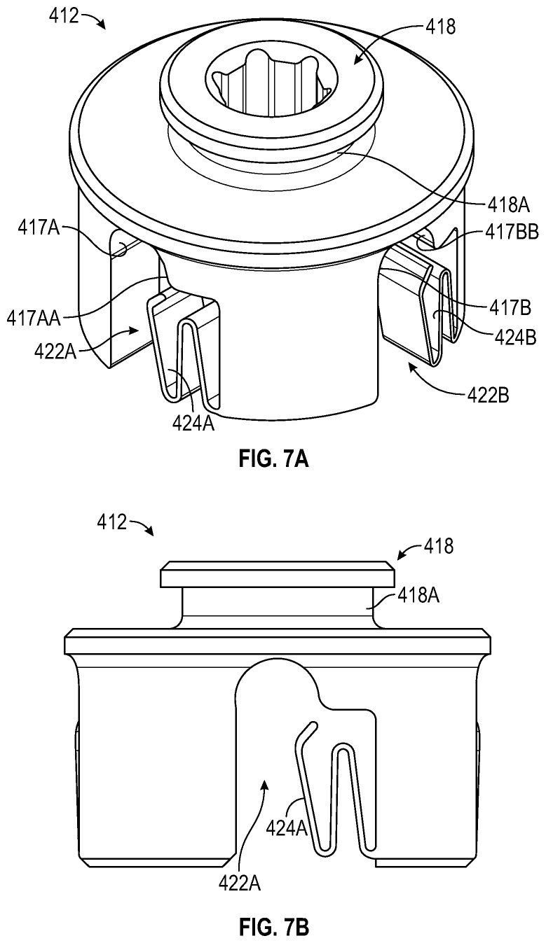

FIG. 7A shows another example of an adapter 412. The adapter 412 can be configured to seat down on and couple with the reamer head 14 (FIG. 1-2C) as in the manner previously described herein and illustrated. The adapter 412 can be configured to positively lock with the reamer head by application of a continuous locking force via a force applied by multiple coupling features 424A and 424B (multiple springs) as further discussed and previously discussed herein.

The adapter 412 can be constructed in a manner similar to that previously described in regards to the adapters 12 (FIGS. 1-3E) and 312 (FIGS. 6A and 6B) described previously. The adapter 412 however differs from these devices in several respects. First, the coupling features 424A and 424B (which can be between two and eight in number) can be located in different orientation than the coupling features described previously. In particular, the coupling feature 424A can be oriented at an angle to the coupling features 424B rather than being aligned therewith as in the case of the example of FIG. 7A. The multiple coupling features 424A and 424B (springs) can be offset from one another such as by being oriented substantially transverse (at 90 degrees) to one another or at another desired angle around the centerline axis of the adapter 412. Thus, the coupling features 414A and 424B are configured to engage different cross-members 30 and 32 of the reamer head 14 shown in FIG. 2.

Additionally, the adapter 412 can include slots 422A and 422B that differ from the configuration of the slots discussed previously. In particular, both the slots 422A and 422B (and optionally in the other two slots not shown) can include modified wall parts. In particular, the slot 422A can include two wall parts 417A and 417AA. The slot 422B can include two wall parts 417B and 417BB. The wall parts 417A and 417B can be hard driving surfaces engaged by the cross-members (now shown) in forward driving rotation of the reamer head. The wall parts 417AA and 417BB can be hard driving surfaces engaged by the cross-members (not shown) in rearward driving rotation of the reamer head.

As shown in FIG. 7B the shape of the coupling feature 424A (coupling feature 424B is not shown but is similarly configured) can be shortened and modified within the slot 422A from the coupling features discussed and illustrated in prior examples. The coupling feature 424A can be configured to engage distal portions of the cross-members (now shown) in a location that differs from those of the coupling features described previously. Put another way, the engagement location of the coupling feature 424A has been clocked relative to the engagement locations of the coupling features of FIGS. 1-3E, 6A and 6B. The coupling feature 424A can slightly modified in shape to provide for increased spring interference and stiffness to the reamer adapter for better stability.

FIGS. 7A and 7B also a first coupling feature 418 that is modified from the first coupling feature 18 described previously. The first coupling feature 418 can include an undercut dovetail 418A or other feature(s) for coupling with a reamer adapter puller or similar instrument that may be utilized for decoupling the adapter from the reamer head.

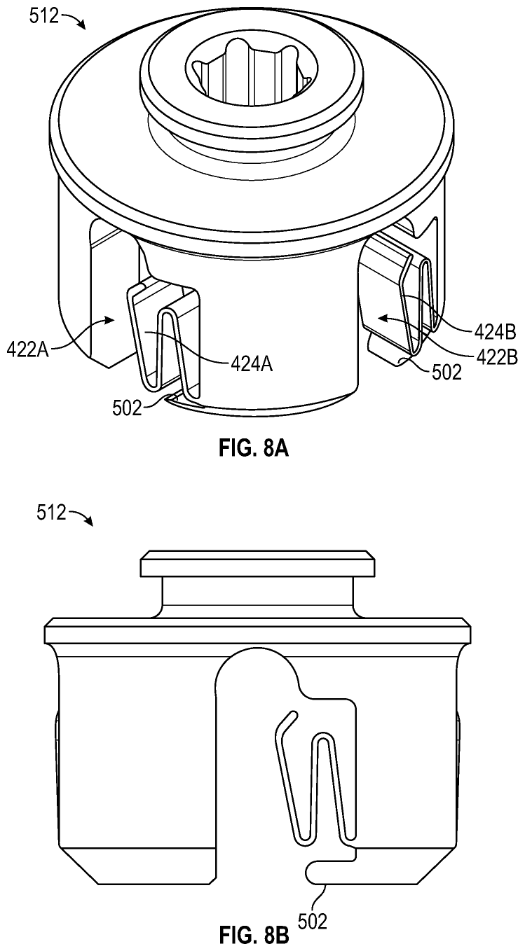

FIGS. 8A and 8B show another example of an adapter 512. The adapter 512 can be configured in the manner of the adapter 412 (FIGS. 8A and 8B) including having the multiple coupling features 424A and 424B and slots 422A and 422B as shown in FIG. 8A. The adapter 512 can be differ from the adapter 412 of FIGS. 7A and 7B in that the adapter 512 can include a guard 502 projecting into the slots 422A and 422B at a distal opening thereto adjacent the respective coupling features 424A and 424B as shown in FIG. 8A. The guard 502 (also shown in FIG. 8B) can be a feature configured to prevent misalignment of the reamer head and/or damage to the coupling features 424A and 424B.

### Examples

Each of the following non-limiting examples (referred to as aspects and techniques below) may stand on its own, or may be combined in various permutations or combinations with one or more of the other examples. Furthermore, any element recited in the examples is optional and not a requirement of the apparatus, system or method.

In some aspects, the techniques described herein relate to an adapter for an orthopedic reamer including: a main body having a recess and a plurality of slots communicating with the recess; and at least a first spring coupled to the main body and positioned in one of the plurality of slots, wherein the first spring is configured to engage a cross-member of a reamer head and force the cross-member to engage with a part of the main body that forms the one of the plurality of slots.

In some aspects, the techniques described herein relate to an adapter, wherein the first spring is formed from the main body such as by 3D printing or wire EDM.

In some aspects, the techniques described herein relate to an adapter, wherein the first spring and the main body are an integral single piece component.

In some aspects, the techniques described herein relate to an adapter, wherein the at least the first spring includes at least two springs that are spaced from one another by another of the plurality of slots.

In some aspects, the techniques described herein relate to an adapter, wherein the plurality of slots include four slots equidistantly spaced about a centerline of the adapter.

In some aspects, the techniques described herein relate to an adapter, wherein the first spring has a plurality of bends and a plurality of sections.

In some aspects, the techniques described herein relate to an adapter, wherein the first spring has an engagement arm configured to engage the cross-member.

In some aspects, the techniques described herein relate to an adapter, further including a coupling feature for connecting the adapter to one of a shaft or a robotic arm.

In some aspects, the techniques described herein relate to an assembly for an orthopedic reamer including: a reamer head having a first cross-member and a second cross-member; an adapter including: a main body having a recess and a plurality of slots communicating with the recess; and at least a first spring coupled to the main body and positioned in one of the plurality of slots, wherein the first spring is configured to engage the first cross-member and force the first cross-member to engage with a part of the main body that forms the one of the plurality of slots; wherein the second cross-member is received in additional of the plurality of slots.

In some aspects, the techniques described herein relate to an assembly, wherein the first cross-member and the second cross-member intersect within the recess.

In some aspects, the techniques described herein relate to an assembly, wherein the first spring is formed from the main body and is an integral single piece component with the main body.

In some aspects, the techniques described herein relate to an assembly, wherein the wherein the at least the first spring includes at least two springs that are spaced from one another by the additional of the plurality of slots.

In some aspects, the techniques described herein relate to an assembly, wherein the first spring has a plurality of bends and a plurality of sections, and wherein the first spring has an engagement arm configured to engage the first cross-member.

In some aspects, the techniques described herein relate to an assembly, wherein the first cross-member and the second cross-member extend in orthogonal directions.

In some aspects, the techniques described herein relate to a robotic surgical system including: an articulatable arm configured to move a distal end of the articulatable arm to a location in a coordinate system for the robotic surgical system; and a surgical instrument coupler connected to the distal end of the articulatable arm, the surgical instrument coupler including: a guide shaft extending from the distal end; an articulation coupler connected to the guide shaft at a fixed location relative to the distal end, the articulation coupler defining a pivot center; an instrument head; an instrument shaft connected to the articulation coupler to pivot about the pivot center; and an adapter coupled to the instrument shaft can configured to couple with the instrument head via one or more springs.

In some aspects, the techniques described herein relate to a robotic surgical system, wherein the adapter is configured to engage with a first cross-member of the instrument head and force the first cross-member to engage with a part of the adapter that forms one of a plurality of slots within the adapter.

In some aspects, the techniques described herein relate to a robotic surgical system, wherein the one or more springs are each positioned within a respective one of the plurality of slots.

In some aspects, the techniques described herein relate to a robotic surgical system, wherein the one or more springs are formed as an integral piece of a main body of the adapter.

In some aspects, the techniques described herein relate to a method of coupling a reamer head to an adapter including: positioning the adapter and reamer head to align a first cross-member with a first plurality of slots of the adapter and a second cross-member to align with a second plurality of slots of the adapter; receiving the first cross-member with the first plurality of slots of the adapter and the second cross-member with the second plurality of slots of the adapter; engaging the first cross-member with one or more springs positioned within one or more of the first plurality of slots; and forcing the first cross-member against a wall of the adapter that defines at least one of the first plurality of slots.

Each of these non-limiting examples can stand on its own, or can be combined in various permutations or combinations with one or more of the other examples.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An adapter for an orthopedic reamer comprising:
a main body having a recess and a plurality of slots communicating with the recess; and
at least a first spring coupled to the main body and positioned in one of the plurality of slots, wherein the first spring is configured to engage a cross-member of a reamer head and force the cross-member to engage with a part of the main body that forms the one of the plurality of slots.

2. The adapter of claim 1, wherein the first spring is formed from the main body.

3. The adapter of claim 1, wherein the first spring and the main body are an integral single piece component.

4. The adapter of claim 1, wherein the at least the first spring comprises at least two springs that are spaced from one another and each positioned in different of the plurality of slots.

5. The adapter of claim 1, wherein the plurality of slots comprise four slots equidistantly spaced about a centerline of the adapter with each slot having a respective one of the at least the first spring.

6. The adapter of claim 1, wherein the first spring has a plurality of bends and a plurality of sections.

7. The adapter of claim 6, wherein the first spring has an engagement arm configured to engage the cross-member.

8. The adapter of claim 1, further comprising a coupling feature for connecting the adapter to one of a shaft or a robotic arm.

9. The adapter of claim 1, further comprising:
a reamer head having a first cross-member and a second cross-member, wherein the second cross-member is received in additional of the plurality of slots.

10. The adapter of claim 9, wherein the first cross-member and the second cross-member intersect within the recess.

11. The adapter of claim 9, wherein the at least the first spring is formed from the main body and is an integral single piece component with the main body.

12. The adapter of claim 9, wherein the at least the first spring comprises at least two springs that are spaced from one another and each positioned in different of the plurality of slots.

13. The adapter of claim 9, wherein the at least the at least the first spring has a plurality of bends and a plurality of sections, and wherein the at least the first spring has an engagement arm configured to engage the first cross-member.

14. The adapter of claim 9, wherein the first cross-member and the second cross-member extend in orthogonal directions and each are engaged by different respective ones of the at least the first spring.

15. A robotic surgical system comprising:
an articulatable arm configured to move a distal end of the articulatable arm to a location in a coordinate system for the robotic surgical system; and
a surgical instrument coupler connected to the distal end of the articulatable arm, the surgical instrument coupler comprising:
a guide shaft extending from the distal end;
an articulation coupler connected to the guide shaft at a fixed location relative to the distal end, the articulation coupler defining a pivot center;
an instrument head;
an instrument shaft connected to the articulation coupler to pivot about the pivot center; and
an adapter coupled to the instrument shaft can configured to couple with the instrument head via one or more springs.

16. The robotic surgical system of claim 15, wherein the adapter is configured to engage with a first cross-member of the instrument head and force the first cross-member to engage with a part of the adapter that forms one of a plurality of slots within the adapter.

17. The robotic surgical system of claim 15, wherein the one or more springs are each positioned within a respective one of a plurality of slots within the adapter.

18. The robotic surgical system of claim 15, wherein the one or more springs are formed as an integral piece of a main body of the adapter.

19. A method of coupling a reamer head to an adapter comprising:
positioning the adapter and reamer head to align a first cross-member with a first plurality of slots of the adapter and a second cross-member to align with a second plurality of slots of the adapter;
receiving the first cross-member with the first plurality of slots of the adapter and the second cross-member with the second plurality of slots of the adapter;
engaging the first cross-member with one or more springs positioned within one or more of the first plurality of slots; and
forcing the first cross-member against a wall of the adapter that defines at least one of the first plurality of slots.

20. The method of claim 19, further comprising engaging the second cross-member with a second one or more springs positioned within one or more of a second plurality of slots.
